(19) United States
(12) Patent Application Publication
(10) Pub. No.: US 2017/0029898 A1
(43) Pub. Date: Feb. 2, 2017

(54) NOVEL METHOD FOR SCREENING FOR PROSTATE CANCER

(71) Applicants: CENTRE NATIONAL DE LA RECHERCHE SCIENTIFIQUE-CNRS, Paris (FR); UNIVERSITE DE MONTPELLIER 1, Montpellier Cx 2 (FR); CENTRE HOSPITALIER UNIVERSITAIRE DE TOULOUSE, Toulouse (FR)

(72) Inventors: Marcel GARCIA, Prades Le Lez (FR); Ophélie VAILLANT, Le Crés (FR); Xavier REBILLARD, Saint Gely Du Fesc (FR); Catherine MAZEROLLES, Toulouse (FR); Magali GARY-BOBO, Castelnau-Le-Lez (FR); Alain MORERE, Saint Gely Du Fesc (FR)

(21) Appl. No.: 15/113,281

(22) PCT Filed: Jan. 22, 2015
(86) PCT No.: PCT/FR2015/050153

§ 371 Date: Jul. 21, 2016

(30) Foreign Application Priority Data
Jan. 23, 2014 (FR) 14 50588

(51) Int. Cl. C12Q 1/68; C07K 16/30; G01N 33/574

(52) U.S. Cl. CPC C12Q 1/6886; G01N 33/57434; C07K 16/3069; C12Q 2600/158

(57) ABSTRACT

The invention relates to a method for the in vitro diagnosis of prostate cancer in a patient, characterised in that it comprises a step of measuring the expression level of the gene of the cation-independent mannose-6-phosphate receptor (CI-M6PR) in a sample of prostate tissue of the patient, the determination of overexpression of said CI-M6PR gene indicating the presence of prostate cancer in said patient.

The subject of the present invention is a novel method for diagnosing or screening for prostate cancer.

Prostate cancer is the most common cancer in France and represents the fourth most common cause of mortality in the population. In reality, individual screening by assaying the level of prostate specific antigen (“PSA”) has become very widespread and 70% of men over the age of 50 have at least one PSA assay over the course of a period of 3 years (2012 figures from the Caisse Nationale de l'Assurance Maladie des Travailleurs Salariés [French National State Health Insurance Office for Salaried Workers]). Seventy thousand new cases of prostate cancer are diagnosed each year in France.

However, two major randomized studies, one American (“PLCO3”: “Prostate, Lung, Colorectal and Ovarian Cancer Screening Trial”), the other European (“ERSPC”, “European Randomized Study of Screening for Prostate Cancer”) have provided contradictory results with regard to the usefulness and benefit of screening by assaying PSA^{1, 2, 3}. The policy report on prostate cancer, published in February 2012 by the Haute Autorité de Santé [French National Health Authority], and a publication in the Bulletin du Cancer² [Cancer Bulletin] demonstrate the imperfect nature of the PSA assay, and of digital rectal examination and the “absence at the current time of a marker and examination for screening and diagnosis making it possible to identify early the forms of prostate cancer”. They also emphasize “the importance of research on effective screening tests and markers for distinguishing the aggressive forms from the indolent forms”.

A prostate cancer lesion is histologically reflected by the loss of basal cells. Currently, antibodies directed against basal cell markers (p63, CK 903, CK 5/6) are used clinically in order to perform the diagnosis. However, their specificity and their sensitivity remain limited, and lesions of noncancerous type (adenoses, atypical adenomatous hyperplasia, atrophy or post-atrophic hyperplasia) can display a discontinuity of basal cells, thereby making interpretation difficult^{5, 6}.

By way of examples of prostate cancer cell markers other than PSA that are envisioned at the current time for diagnosis, mention may be made of AMACR®, methylated GSTP1® and TMPRSS2-ETS®. AMACR® (P504S) is the trade name of an antibody specific for the tissue protein “α-methylacyl CoA racemase” which is hyperexpressed in prostate cancers'. This antibody was thus developed in order to analyze the expression profile in prostate biopsies.

However, said markers (AMACR®, methylated GSTP1®, TMPRSS2-ETS®) have limits since they are also overexpressed in benign prostatic hyperplasia⁸. Furthermore, some prostate cancers are not diagnosed with these markers. Thus, no consensus has been found at the current time for their clinical use.

Prostate cancer is currently diagnosed by anatomopathological examination of a series of prostate biopsies (10 to 12 cores 17 mm long, obtained using an 18-gauge needle). The samples are evenly distributed under echographic control over the prostate volume in order to perform mapping. Prostate cancer is heterogeneous and cancerous foci can emerge in one or other prostate lobe or both. The diagnosis of prostate cancer is dependent on the quality of the sample and on the modes for taking said sample. Furthermore, despite the histological observation and the analysis of basal cell markers, some noncancerous tissue lesions may mimic a cancer diagnosis, or conversely, a cancerous lesion may escape diagnosis, thereby requiring the addition of tissue markers specific for prostate cancer cells.

It is therefore of great advantage to be able to have new specific tools for facilitating diagnosis, estimation of tumor aggressiveness and individual risks of progression in order to direct the choice of treatment of the patient and to avoid allowing cancerous lesions to progress.

One of the objectives of the present invention is therefore to provide a method for the diagnosis of prostate cancer which does not have the drawbacks of those provided to date.

Another objective of the invention is to provide a method for the specific diagnosis of prostate cancer.

Another objective of the present invention is to diagnose prostate cancer when the latter is at an early stage.

Another objective of the invention is to diagnose prostate cancer of small size.

The present invention results from the surprising and unexpected discovery of the overexpression, at tissue level, of the cation-independent mannose-6-phosphate receptor (referred to hereinafter as CI-M6PR) in cancerous epithelial cells of the prostate compared with healthy prostate cells.

Indeed, against all expectations, the inventors have determined that CI-M6PR is overexpressed in a vast majority of cancerous prostate tissues, but that it is not overexpressed in healthy prostate tissues.

This unexpected discovery makes it possible to envision the value of CI-M6PR as a specific marker for the purpose of diagnostic use.

CI-M6PR is a ubiquitous receptor of 300 kDa present at the surface of cells. It is involved in numerous biological functions and mainly in the trafficking of enzymes to the lysosome⁹. These lysosomal enzymes have mannose 6-phosphate (M6P) residues which allow them to be recognized and then internalized by CI-M6PR.

CI-M6PR is also known as the insulin growth factor 2 receptor (IGF2) since it has an IGF2-binding site and is involved in the degradation of this mitogen⁴. Thus, CI-M6PR and IGF2R, also referred to hereinafter as “CI-M6PR gene”, “CI-M6PR receptor”, or “IGF2R gene” or “IGF2R receptor”, have the same nucleotide sequence and are in reality the same gene (gene identified by number “3482” on Jan. 15, 2015). The genomic sequence of CI-M6PR (or IGF2R) is located on chromosome 6 and is described under NCBI accession number NG_011785.1. The corresponding mRNA sequence is described under NCBI accession number NM_000876.2 and the protein sequence under accession number NP_000867.2.

During carcinogenesis, the expression profile of many genes and proteins is modified. In previous studies carried out on multiple cancers, CI-M6PR was characterized as a tumor suppressor gene.

In liver, breast, lung or ovarian cancers or else adenocortical tumors, the CI-M6PR receptor exhibits a loss of heterozygosity and numerous somatic mutations^{10,11,12}. Post-transcriptional and post-translational modifications lead to a decrease in expression of the CI-M6PR receptor in these cancers, except for breast cancer, where its concentration is unchanged between healthy and cancerous tissues¹³. In certain cancerous cells, the tumor suppressor role for the CI-M6PR gene has been proposed by virtue of experiments in which there is induction of overexpression¹⁴ or, conversely, inhibition of the expression of CI-M6PR¹⁵. In the first case, the overexpression of CI-M6PR decreases the capacity of the cells to induce tumors and decreases the rate of tumor growth. Conversely, the inhibition of CI-M6PR increases cell growth and decreases the apoptotic index.

The presence of anti-CI-M6PR antibodies has been shown by Huang et al.¹⁶ in 7 patients suffering from prostate cancer out of 23. However, this result could not direct those skilled in the art toward looking for overexpression of CI-M6PR originating from cancerous tissue for two reasons: firstly, as previously described, CI-M6PR was considered to be a tumor suppressor gene, the expression of which was therefore decreased in cancers, and secondly it was clearly published in the prior art that the best circulating prognostic marker, PSA, was produced by healthy prostate cells and more weakly by cancerous cells^{∫}.

After numerous studies, the inventors have overcome a technical prejudice by showing that the CI-M6PR gene constitutes a relevant marker in screening for or diagnosing prostate cancer.

The use of the novel marker of the invention, namely CI-M6PR, is specific for prostate cancer and as a result is of great value for the diagnosis of said cancer.

A subject of the present invention is a method for the in vitro diagnosis of prostate cancer in a subject, characterized in that it comprises a step of measuring the expression level of the CI-M6PR gene in a sample of prostate tissue from said subject, the determination of overexpression of said CI-M6PR gene being an indication of the presence of prostate cancer in said subject.

The “method for the diagnosis” of the invention may also be referred to as “process for the diagnosis”.

In one particular embodiment, the method of the invention is carried out for the diagnosis, prognosis and/or evaluation of the progression of prostate cancer in a subject, overexpression of the CI-M6PR gene in the prostate tissue being an indication of prostate cancer.

The term “diagnosis” is intended to mean the determination of an ailment of an individual suffering from a given pathological condition, and the term “prognosis” is intended to mean the evaluation of the degree of seriousness and of the subsequent progression of a pathological condition.

The term “overexpression of the CI-M6PR gene” is intended to mean expression of the CI-M6PR gene at prostate tissue level which is at least three times greater than the expression of this gene at the level of a noncancerous prostate tissue.

In this case, when the expression of the CI-M6PR gene is increased by a factor of at least 3 in a sample of prostate tissue, then it may be concluded that said sample of prostate tissue is a sample of cancerous prostate tissue; in other words, the subject to whom said sample belongs is suffering from prostate cancer.

This overexpression will be at least three times greater, but may for example be ten, twenty or even one hundred times greater compared with the expression of the CI-M6PR gene in a “normal”, i.e., noncancerous, prostate tissue.

More particularly, a subject of the present invention is a method for the in vitro diagnosis of prostate cancer in a subject, characterized in that it comprises the following steps:
(i) of quantitative measurement of the expression level of the CI-M6PR gene in a sample of prostate tissue from said subject;
(ii) of comparison of said expression level of said subject with the expression level of the CI-M6PR gene of a reference biological sample.

When the expression level of CI-M6PR is quantitatively higher by a factor of at least 3 in the sample of prostate tissue from the subject to be diagnosed than in the reference biological sample, a conclusion of prostate cancer can be reached.

A higher expression level signifies, for example, a protein expression (measured by various techniques such as Western blotting, ELISA, biosensors, receptor-specific ligands or any method for quantifying a protein or a receptor) which is at least 3 times greater than that of a reference biological sample corresponding for example to healthy prostate tissue.

In addition to the quantitative evaluation as described above, it is also possible to evaluate the overexpression of the CI-M6PR gene in a prostate tissue by evaluating the percentage of cells of said prostate tissue which are stained using an immunohistological test. Thus, in a specimen of prostate tissue, the presence of a 3-fold increase in the immunohistochemical labeling in more than 10% of the epithelial cells is an indication of a cancerous prostate tissue.

For the purposes of the present invention, the term “subject” denotes a vertebrate individual, in particular a mammal, more particularly a man.

For the purposes of the present invention, the term “sample of prostate tissue” is intended to mean all or part of the prostate taken from a subject or a patient. The prostate tissue comprises prostate cells, said cells possibly being cancerous cells or healthy cells.

This sample of prostate tissue is obtained by means of any type of sampling known to those skilled in the art. According to one preferred embodiment of the invention, the sample of prostate tissue taken from the individual or the patient is from a prostate biopsy.

The “reference biological sample” is obtained from human cell cultures or from tissue specimens. For the purposes of the present invention, the reference biological sample denotes a biological sample as defined above, from:
a human cell line (normal or cancerous) in culture in which the expression of the CI-M6PR gene is known to be low,
a healthy subject, namely not having prostate cancer,
a subject in prostate cancer remission,
a subject in whom the diagnosis of cancer is to be established by means of multiple biopsies and in whom one or more of these biopsies exhibit(s) a low expression of the CI-M6PR gene, or
a subject in whom the expression of the CI-M6PR gene is known and associated with a particular clinical stage.

In the latter case, the invention allows the follow-up and monitoring of a patient suffering from prostate cancer and undergoing anticancer treatment.

The method according to the invention is further characterized in that the step of measuring the expression level of the CI-M6PR gene is a step of measuring, in a sample of prostate tissue, the expression level:
of the transcription products, in particular the mRNA and/or
of the translation products, in particular the CI-M6PR protein.

Overexpression of the transcription products, in particular the mRNA, and/or overexpression of the translation products, in particular the CI-M6PR protein, is then an indication of prostate cancer, said overexpression being as defined previously (increase by a factor of at least three in the expression level of the transcription and/or translation products).

The measurement of the expression level of the CI-M6PR target gene can be carried out by any technique known to those skilled in the art.

According to one advantageous embodiment of the invention, the expression level of the CI-M6PR gene is advantageously measured at the nucleic and/or protein level, for example by measuring the amount of mRNA transcribed and/or by measuring the amount of CI-M6PR protein using at least one specific method for measuring the expression level of CI-M6PR.

The techniques for detecting the expression of the CI-M6PR gene at the nucleic level are well known to those skilled in the art. The detection can in particular be carried out by real-time quantitative RT-PCR, a microfluidic technique, a DNA chip, high-throughput sequencing of the mRNAs, or any appropriate technique for quantifying mRNA, such as an RNA chip, or LCR (ligase chain reaction), TMA (transcription mediated amplification), PCE (enzyme amplified immunoassay) and bDNA (branched DNA signal amplification), etc., methods.

The techniques for detecting the expression of the CI-M6PR gene at the protein level are also well known to those skilled in the art and may in particular include flow cytometry, semi-quantitative immunohistochemistry, quantitative immunocytochemistry, cell ELISA, Taqman(R) protein assay (Applied Biosystems), protein or antibody chips optionally coupled to mass spectrometry, ligand binding, detection on biosensors, detection using optical fibers, etc.

According to the invention, in the method for in vitro diagnosis as defined above, the overexpression of the CI-M6PR gene, and in particular the overexpression of the transcription products and/or of the translation products of the CI-M6PR gene, is determined (established) when the expression of said CI-M6PR gene is at least three times greater than that of said gene in a noncancerous prostate tissue.

According to the invention, in the method for in vitro diagnosis as defined above, the step of measuring the expression level of the translation products of the CI-M6PR gene, in particular the CI-M6PR protein, is carried out by analysis of the immunohistochemical labeling of said CI-M6PR translation products in said sample of prostate tissue from said subject.

More particularly, the analysis of the immunohistochemical labeling of the translation products is evaluated by staining the cells of the sample of prostate tissue, a staining of more than 10% of said cells being an indication of overexpression of said translation products of said gene.

The immunohistochemical labeling allows specific detection of the CI-M6PR gene on cytological material or on tissue sections. Typically, this step is carried out on a chip. Preferentially, said chip is a chip of Tissue Multi Array type.

The use of a chip in diagnosis allows, on a miniaturized format of the size of a glass slide, the analysis and visualization of molecular targets in a large number of tissue samples simultaneously, at the

DNA, RNA or protein level. Typically, the use of a chip makes it possible to obtain expression profiles by immunohistochemistry (IHC) using paraffin-embedded, fixed archived tissues, but also using fresh or frozen tissues. The use of such a chip falls within the normal competence of those skilled in the art.

More particularly, the step of measuring the expression level of the CI-M6PR protein, in a sample of prostate tissue, is carried out using the IgY 415 polyclonal antibody.

This IgY 415 antibody was established in chickens¹⁸ and previously described for immunohistochemical analysis¹⁹.

20

A further subject of the invention is a CI-M6PR-specific antibody for use thereof in a method for the in vitro diagnosis of prostate cancer in a subject, said antibody making it possible to measure the expression level of the CI-M6PR gene in a sample of prostate tissue from said subject, the determination of overexpression of said CI-M6PR gene being an indication of the presence of prostate cancer in said subject.

Thus, as previously mentioned, the expression of the

CI-M6PR gene may for example also be detected by ligand binding. For example, the specific binding of mannose 6-phosphate analogs to CI-M6PR receptors can be quantified⁹. Moreover, this CI-M6PR receptor is a multifunctional protein since it is also the specific receptor for IGF2 (insulin-like growth factor 2)⁴. The binding of IGF2 to its specific site of very high affinity constitutes an effective means for quantifying the expression of this CI-M6PR receptor in prostate tissue.

The expression of the CI-M6PR gene can also be detected using biosensors, either on prostate tissues sampled by means of biopsies, or directly in situ on prostate tissues of the subject.

For in situ detection, these biosensors may, by way of example, consist of optical fibers introduced into the prostate by means of techniques known to those skilled in the art. It is also possible, for in situ detection, to intravenously inject nanoparticles into the subject to be diagnosed. Once injected, these nanoparticles will preferentially accumulate in the cancerous tissue.

A subject of the present invention is also a method for the in vivo diagnosis of prostate cancer in a subject, characterized in that it comprises a step of measuring the expression level of the cation-independent mannose 6-phosphate receptor gene (CI-M6PR gene) in the prostate tissue of said subject, the determination of overexpression of said CI-M6PR gene in said prostate tissue being an indication of the presence of prostate cancer in said subject.

In such a method of diagnosis, the step of measuring the expression level of the CI-M6PR gene in the prostate tissue will for example be a step of measuring the expression level of the translation products, in particular the CI-M6PR protein in said tissue.

A further subject of the invention is a CI-M6PR-specific antibody for use thereof in a method for the in vivo diagnosis of prostate cancer in a subject, said antibody making it possible to measure the expression level of the CI-M6PR gene in the prostate tissue of said subject, the determination of overexpression of said CI-M6PR gene being an indicator of the presence of prostate cancer in said subject.

Another subject of the invention relates to the use of a kit comprising at least one reagent specific for the product of the expression of the CI-M6PR gene, preferably chosen from:
a monoclonal or polyclonal antibody;
a natural or synthetic ligand of CI-M6PR;
any other type of molecules or macromolecules capable of specifically interacting with the CI-M6PR protein;
a nucleic sequence capable of specifically hybridizing with a fragment of the mRNA encoding CI-M6PR,

for the diagnosis and/or prognosis and/or evaluation of the progression of prostate cancer in a subject.

A further subject of the present invention is a method for the therapeutic treatment of prostate cancer in a subject, characterized in that it comprises:
A) a method for the in vitro or in vivo diagnosis of prostate cancer in a subject, comprising a step of measuring the expression level of the CI-M6PR gene, the determination of overexpression of said CI-M6PR gene being an indication of the presence of prostate cancer in said subject,
B) treatment of the cancer thus diagnosed.

Typically, step B) is a conventional step of prostate cancer treatment. By way of example, mention may be made of:
surgery,
radiotherapy (such as external radiotherapy and curietherapy),
hormone therapy,
chemotherapy,
high intensity focused ultrasound therapy,
cryotherapy,
prostatectomy,
photodynamic therapy.

The choice of the therapeutic strategy carried out in step B) depends, inter alia, on the characteristics of the cancer, as diagnosed in step A).

Practioners may also consider other criteria, such as:
the size of the tumor,
whether or not cancerous cells are present in the neighboring lymph nodes, and/or
the presence or absence of metastases in other parts of the body,
the presence of androgen receptors or of other biomarkers (PSA, AMACR®, etc.).

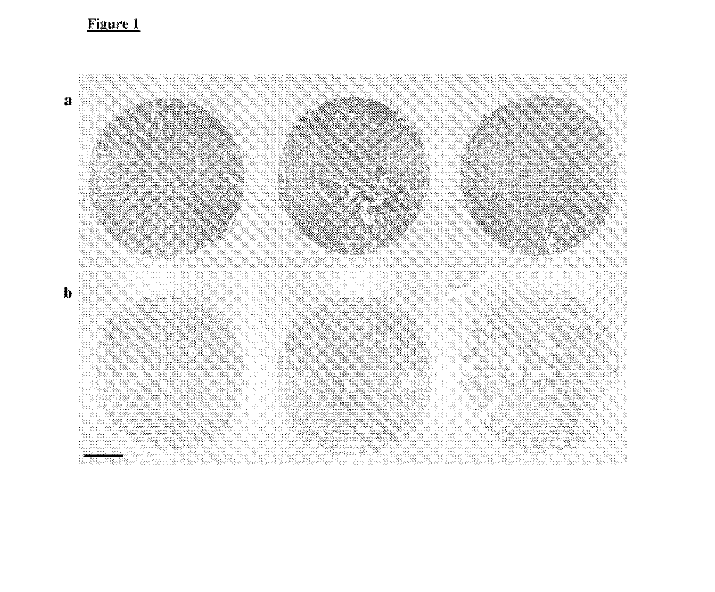

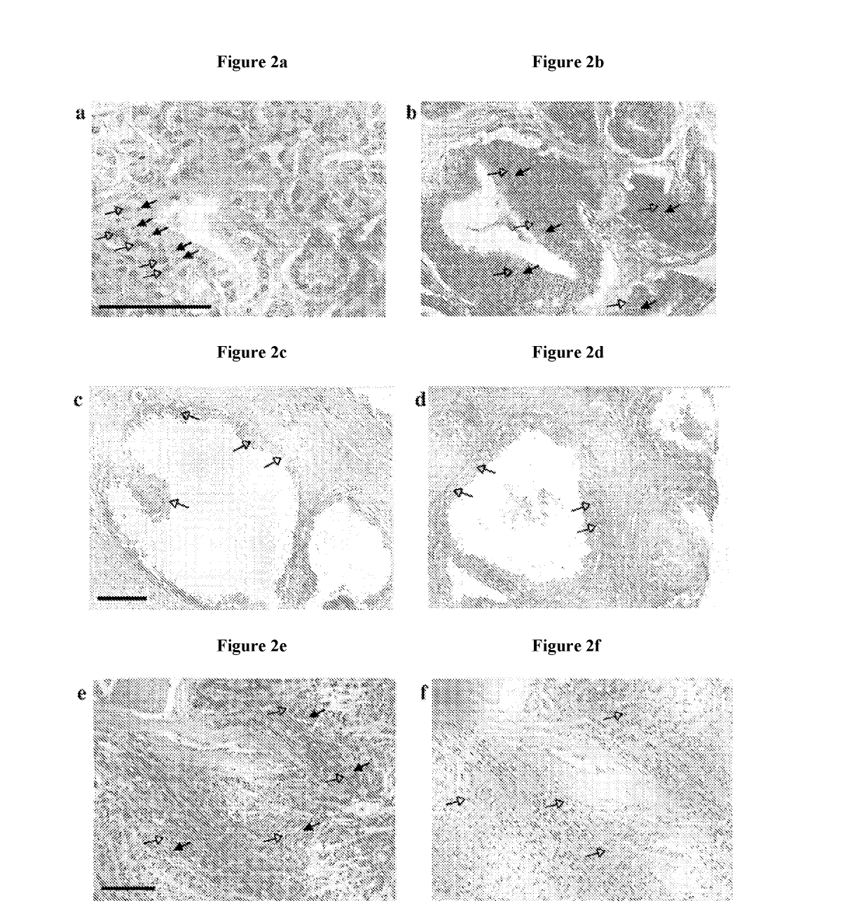

The invention will be understood more clearly in the light of the following nonlimiting and purely illustrative examples, and of FIGS. 1 and 2.

FIG. 1 illustrates the expression profiles of the CI-M6PR receptor in various cancerous prostate tissues (FIG. 1a) and in healthy prostate tissues (FIG. 1b). The scale indicated represents 500 μm.

FIG. 2 shows two types of immunohistochemical labeling of the CI-M6PR gene. The scale indicated represents 100 μm.

FIGS. 2a and 2b illustrate the overexpression of

CI-M6PR in the cancerous prostate tissues owing to their labeling (staining symbolized by solid arrows) using the anti-CI-M6PR antibody IgY 415. The cell nuclei are indicated by hollow arrows.

FIGS. 2c and 2d illustrate healthy prostate tissues.

FIGS. 2e and 2f illustrate the immunohistochemical analysis of two parts of the same sample in the absence (FIG. 2e) or in the presence (FIG. 2f) of an excess of purified CI-M6PR in order to block the immunolabeling.

### EXAMPLE 1

### Study of the Tissue Expression of CI-M6PR Using an Immunohistochemical Method in Paraffin-Embedded Samples

This example describes, in detail, the “tissue multi array” (TMA) technique for immunohistochemical labeling of CI-M6PR on slides of cancerous or healthy prostate tissues. Indeed, the tissue nature of the novel CI-M6PR marker of the invention requires analysis by immunolabeling of prostate biopsies according to the same protocol as that used on TMA slides.

The biopsies are taken routinely and finalize the diagnosis once the digital rectal examination is suspicious or the total PSA level is above 4.

The urologist performs, beforehand, an endorectal echography in order to localize the site where the biopsy will be performed, said biopsy then being taken in the form of a series of 10 to 12 (or more if necessary) specimens using an automatically triggered needle.

The duration of the sampling is very short (5-15 minutes) and the examination not very painful. Complications occurring after a biopsy are rare and antibiotic treatment is carried out as prevention of any infection.

The anatomopathologist then examines the biopsies by histopathological examination in order to establish whether or not cancerous cells are present and/or by immunohistochemical examination in order to study the expression of the CI-M6PR marker sought.

1. Labeling Protocol

The polyclonal antibody called “anti-CI-M6PR IgY 415” or more simply “IgY 415” in what follows is used in order to measure the expression levels of CI-M6PR in human prostate tissues.

This high-affinity antibody purified from eggs of a chicken immunized with CI-M6PR, is specifically directed against the human CI-M6PR receptor¹⁸. The specificity of this antibody for the CI-M6PR receptor has been previously demonstrated by immunohistochemical studies in breast cancers¹⁹.

The analysis of the prostate tissue expression was carried out by immunohistochemical labeling of CI-M6PR on eight TMA slides originating from the pathology department of the CHU [University Hospital Center] of Toulouse (Dr Catherine Mazerolles). Each of these slides comprises 16 cancerous or healthy prostate samples belonging to 8 different patients (2 samples per patient).

The tissues are deparaffinized, rehydrated and then treated with pronase at 0.1% in a phosphate saline solution (PBS) for 10 min at 37° C. Between each step, the samples are washed with a solution of PBS-Tween 20 at 0.1%. The endogenous peroxidases are blocked using a 1% aqueous hydrogen peroxide solution for 15 min. The slides are then incubated for 30 min at 37° C. with a solution of PBS+0.5% bovine gamma globulin (BGG)+goat serum diluted to 1/40 in order to saturate the nonspecific sites. The IgY 415 primary antibody is diluted to 1/1800 in a solution of PBS+0.5% BGG and incubated on the tissues overnight at 4° C.

The IgY 415 antibodies specifically bound to the CI-M6PR are revealed using a rabbit anti-chicken polyclonal secondary antibody coupled to peroxidase (Sigma). The secondary antibody diluted to 1/300 is incubated for 30 min at ambient temperature. Finally, a substrate of the peroxidase, namely 3,3′-diaminobenzidine tetrachloride (Sigma, Saint Quentin Fallavier, France), is incubated for 20 min and precipitation thereof results in a brown/chestnut brown precipitate. The samples are counterstained with hematoxylin and then dehydrated again.

The cell nuclei are thus stained blue/violet by the hematoxyline and are symbolized in FIG. 2 by hollow arrows. The overexpression of CI-M6PR is indicated by the chestnut brown staining and is symbolized in FIG. 2 by solid arrows.

2. Quantification of the Immunolabeling

The slides were scanned on a Nanozoomer-XR (Hamamatsu) and the immunohistochemical labeling of CI-M6PR was analyzed.

The results are expressed as a function of the percentage of cells stained and of the type of staining (perinuclear or dispersed in the cytoplasm).

The tissue analyzed is considered to be positive (i.e. cancerous tissue) if more than 10% of the cells are stained chestnut brown.

All the tissue sections are analyzed and quantified by two different pathologists.

3. Results of the Analysis of the Immunohistochemical Labeling of the CI-M6PR

In total, a collection of 167 human prostate samples collected by prostatectomy and fixed in paraffin were selected by an anatomopathologist. Among them, 126 samples were clearly identified as cancerous tissues and 41 were identified as noncancerous tissues on the basis of their histological analysis. The noncancerous samples comprise 39 normal prostate tissues and 2 benign hypertrophies.

Each sample was both analyzed by a pathologist in order to determine the Gleason scores corresponding to the seriousness of the cancer and immunostained using an anti-M6PR antibody (IgY 145 antibody). The cell nuclei were stained with hematoxylin.

FIG. 1 illustrates the expression profiles of the CI-M6PR receptor in three different prostate cancers (FIG. 1a) and in three healthy prostate tissues (FIG. 1b). The difference in immunohistochemical labeling of CI-M6PR between the healthy and cancerous tissues is very considerable at this magnification. In the healthy tissues, only the labeling of the nuclei with hematoxylin is visible.

FIG. 2 shows, at a high magnification, the characteristics of the immunohistochemical labeling with the anti-CI-M6PR antibody. The cancerous prostate tissues are labeled differently since the staining of the prostate cancer tissues indicated two strong types of labeling (two expression profiles) considered to be positive (FIGS. 2a and 2b).

The first type of staining, observed in 23% of the samples, is granular and perinuclear. This staining is the most standard (FIG. 2a) and is generally observed in normal tissues rich in M6PR^{13, 14, 15}.

The second type of staining, observed in 61% of the samples, is also granular, but more diffused in the cytoplasm of the cell (FIG. 2b).

These two types of labeling indicate overexpression of CI-M6PR.

On the other hand, the epithelial cells are not stained in the normal prostate tissues, thereby suggesting that the overexpression is specific for malignant cells (FIG. 2c, 2d).

In conclusion with respect to FIGS. 1 and 2, it is noted, surprisingly, that the epithelial cells of the healthy prostate tissues are not labeled with the antibody, these tissues therefore express little CI-M6PR, while the cancerous prostate tissues overexpress the CI-M6PR receptor.

Table 1 below indicates the percentage of labeled cells in cancerous and healthy prostate tissues.

TABLE 1

| | Cancerous | Noncancerous |
|---|---|---|
| % of labeled | prostate tissue | prostate tissue |
| CI-M6PR cells | (126) (%) | (41) (%) |
| 0-3% | 0 (0) | 41 (100) |
| 3-10% | 20 (16) | 0 (0) |
| 10-30% | 28 (22) | 0 (0) |
| 30-60% | 39 (31) | 0 (0) |
| 60-100% | 39 (31) | 0 (0) |

As a reminder, a level greater than 10% of labeled cells was set by the inventors as an indicator of a cancerous prostate tissue. The labeled cells are also denoted “labeled CI-M6PR cells” or “stained CI-M6PR cells”.

Out of 126 cancerous prostate samples, 106 samples in total—i.e. 84% of cancerous prostate samples exhibit more than 10% of labeled cells.

On 31% of cancerous prostate samples, more than 60% of the cells are labeled. In contrast, on all of the 41 healthy samples, less than 3% of the cells are labeled.

Table 2 below indicates the overexpression of the CI-M6PR receptor (% CI-M6PR positive) in 126 cancerous prostate samples as a function of the Gleason grade. The term “% CI-M6PR positive” indicates the percentage of samples having a level of cells labeled for CI-M6PR which is greater than 10%.

The seriousness and the risk of progression of the prostate cancers are estimated in particular by means of the Gleason grade, also called Gleason score (score ranging from 2 to 10) taking into account the modification of the morphology of the prostate glands toward an increasing undifferentiation.

TABLE 2

| Gleason grade (number of samples) | % CI-M6PR positive |
|---|---|
| Grade 4 (2) | 100 |
| Grade 5 (8) | 75 |
| Grade 6 (30) | 90 |
| Grade 7 (56) | 80 |
| Grade 8 (23) | 83 |
| Grade 9 (6) | 100 |
| Grade 10 (1) | 100 |
| Total: 126 samples | 84% |

The analysis of the immunohistochemical labeling of CI-M6PR according to the Gleason scores indicates that CI-M6PR is overexpressed (positive staining) both for low grades of cancer (such as grade 4) and for high grades of cancer (such as grade 10).

In conclusion, the immunohistochemical analysis of 126 prostate cancers shows overexpression of CI-M6PR in 84% of cases, whereas the overexpression of this receptor is nondetectable in 41 healthy or benign hyperplastic prostate biopsies.

These results make it possible to envision CI-M6PR for use thereof as a biomarker in the diagnosis of prostate cancer in a subject.

4. Specificity of the Immunolabeling

In order to validate the specificity of the immunolabeling and the results obtained, an immunohistochemical labeling reversion experiment was carried out by prior saturation of the IgY 415 antibody with an excess of CI-M6PR antigen.

Samples of cancerous prostate tissues, supplied by Dr Xavier Rebillard (Beausoleil Clinic, Montpellier), were fixed with 4% paraformaldehyde, embedded in paraffin and sectioned on a Leica microtome (Leica Biosystems) in sections 5 μm thick. Two consecutive sections of the same sample were used. The labeling protocol remains identical, but the IgY 415 antibody (1/1000) is incubated beforehand for 90 min at 37° C. with a highly concentrated CI-M6PR solution (1.24 mg/ml) in PBS or with a PBS solution containing 1.24 mg/ml of bovine gamma-globulin (control).

The analysis of the two conditions made it possible to validate the specificity of the CI-M6PR labeling since the saturation of the IgY 415 antibody with an excess of CI-M6PR receptor blocked the immunohistochemical staining (FIG. 2f) observed on the control slide (FIG. 2e).

### EXAMPLE 2

### Immunohistochemical Labeling of CI-M6PRs by Immunohistochemistry of Frozen Cancerous Biopsies and of Frozen Normal Tissue

The biopsies are from prostatectomies carried out for the treatment of advanced cancers. The tissue sampled is frozen at −80° C. and then fixed by incubation for 20 seconds in methanol at a temperature of −20° C. The 6 μm frozen sections are prepared and incubated for 30 min at ambient temperature with the antibodies (dilution 1/300) in the phosphate buffer.

The expression level of CI-M6PR was measured using a computerized image analyzer (SAMBA, Alcatel Grenoble France) according to the method described in Berthe¹⁹.

The results are expressed in “arbitrary values” obtained by the formula of the QIC score (Quantitative Immuno Cytochemical score)=(percentage of stained surface of the epithelial cells)×(mean staining intensity)×10.

The specific labeling is obtained using the IgY 415 antibody (dilution 1/1000).

The nonspecific labeling is evaluated using an identical concentration of an IgY antibody originating from a nonimmunized animal (negative control).

Table 3 below describes the expression level of CI-M6PR in frozen sections of 5 specimens according to the determination of the QIC score with the M6PR antibody IgY 415 or the control (nonspecific) antibody.

TABLE 3
Assay of the expression level of CI-M6PR on frozen
sections originating from three specimens of prostate
cancer and from two specimens of normal prostate
tissues by the QIC score method.

| | Anti-CI-M6PR | Nonspecific |
|---|---|---|
| | antibody | antibody |
| | QIC score | QIC score |
| Prostate | (mean ± standard | (mean ± standard |
| cancers | deviation) | deviation) |
| Patient 1 | 95 ± 15 | 20 ± 4 |
| Patient 2 | 85 ± 10 | 17 ± 5 |
| Patient 3 | 110 ± 10 | 18 ± 5 |
| Normal tissue 1 | 22 ± 5 | 19 ± 6 |
| Normal tissue 2 | 22 ± 5 | 22 ± 4 |

These results show quantifiable and intense expression in the cancerous cells.

In the cancerous tissues, the immunohistochemical labeling is 3.4 to 5 times higher than that of the normal tissues. The labeling of the cancers that is obtained with the anti-CI-M6PR antibody is specific since it is 4.75 to 6 times higher than that obtained for the same concentration of a nonspecific IgY antibody.

The assaying by immunohistochemistry can therefore be carried out on frozen sections of the prostate tissue. This type of section advantageously makes it possible to establish a rapid (early) diagnosis after sampling by the clinician. Thus, according to the invention, an “extemporaneous” diagnosis is obtained, which takes place immediately after sampling of prostate tissue from a subject to be diagnosed.

Conclusion

The expression of CI-M6PR has previously been observed in isolated cancer cell lines in culture, including in a prostate cancer line¹⁶. It has thus been shown, in Huang et al.¹⁶, that the LNCaP prostate cancer cell line expresses CI-M6PR, whereas the other prostate cancer cell lines PC-3 and DU-145 express very little or no CI-M6PR.

However, labeling on an isolated prostate cancer cell line can in no way lead to the prediction of overexpression of CI-M6PR in all cancerous prostate tissues. This is because a cell line is an artificial system subjected to culture artifacts, for instance the presence of fetal calf serum essential for survival of the cell line, and therefore the results obtained with a cell line cannot be generalized, all the more so if the results obtained with other comparable cell lines are different.

Moreover, as previously described, CI-M6PR was considered to be a tumor suppressor gene since many studies described a decrease in the expression of CI-M6PR in several types of cancers.

To finish, those skilled in the art also knew that the best circulating prognostic marker, PSA, was produced essentially by healthy prostate cells.

Thus, by virtue of the teaching of the prior art, no analysis of the expression of CI-M6PR and comparison of the tissue of a “healthy” prostate and of a “cancerous” prostate had been carried out to date.

The results of the overexpression of CI-M6PR in cancerous prostate tissues are therefore very unexpected since they go against the knowledge of the prior art and make it possible to overcome a technical prejudice.

The overexpression of CI-M6PR in cancerous prostate tissues makes it possible to envision the use of said CI-M6PR as a diagnostic tool for screening for prostate cancers by anatomopathologists.

The absence of overexpression of CI-M6PR by healthy prostate tissues advantageously excludes the recurrent problems of false positives and of overdiagnosis observed with other potential markers (for instance AMACR)⁷.

This new marker CI-M6PR for screening for and for the diagnosis of prostate cancer according to the invention therefore advantageously makes it possible to overcome the deficiencies and the limits of the current diagnostic tools.

The present invention provides a marker specific for prostate tumor cells since CI-M6PR is overexpressed only in the cancerous cells of prostate tissues (and not in healthy prostate tissues).

The present invention also provides a novel marker for cancerous cells which can be measured on the prostate tissue in situ in the patient by means of methods using biosensors of optical fiber or nanoparticle type.

In order to supplement the anatomopathological diagnosis, the method of the invention may further comprise, in addition to the measurement of the expression level of the CI-M6PR gene, the measurement of the expression level of one or more immunohistochemical markers known at the current time.

By way of example of immunohistochemical markers, mention may be made of basal cell markers (also known as “basal marker”), in particular chosen from p63, CK 903 or CK 5/6, or other cancerous cell markers, in particular chosen from AMACR®, methylated GST1® or TMPRSS2-ETS®.

### LITERATURE REFERENCES

1. Andriole G. L., et al J Nat Cancer Inst 104: 1-8 (2012).

2. Schröder F. H. et al., N Engl J Med 360: 1320-1328 (2009).

3. Schröder F. H. et al., N Engl J Med 366: 981-990 (2012).

4. Valeri A. et al., Bull Cancer 12: 1499-1515 (2010).

5. Molinié V. et al., Prog Urol 4: 611-615 (2005).

6. Makarov D. V. et al, Annu Rev, Med 60: 139-151 (2009).

7. Rubin M. A. et al. Jama 287: 1662-1670 (2002).

8. Chikezie O. et al J Cancer 1: 150-177 (2010).

9. Gary-Bobo M. et al., Curr Med Chem 14 (28): 2945-2953 (2007).

10. Hebert E., Biosci Rep 26: 7-17 (2006).

11. Da Costa S. A. et at. J Mammary Gland Biol Neoplasia 5: 85-94 (2000).

12. Yamada T. et al., Proe Natl Acad Sci USA 94: 10351-10355 (1997).

13. Hebert E. et al., Br J Cancer 69: 120-124 (1994).

14. Lee J. S. et at., Int J Cancer 107: 564-570 (2003).

15. Chen Z. et at., BMC Cancer 2: 18-27 (2002).

16. Huang Y. et al., Clin Immunol 111: 202-209 (2004).

17. Leong A. S. Y. et al., Manual of Diagnostic Cytology, 2nd edition, Greenwish Medical Media. 79-80 (2003).

18. Lemamy G. et al., Int J Cancer 80: 896-902 (1999).

19. Berthe M. L. et al., Eur J Cancer 39: 635-642 (2003).

1. A method for the in vitro diagnosis of prostate cancer in a subject, wherein it comprises a step of measuring the expression level of the cation-independent mannose 6-phosphate receptor gene (CI-M6PR gene) in a sample of prostate tissue from said subject, the determination of overexpression of said CI-M6PR gene being an indication of the presence of prostate cancer in said subject.

2. The method as claimed in claim 1, wherein the step of measuring the expression level of the CI-M6PR gene is a step of measuring the expression level of the transcription products.

3. The method as claimed in claim 1, wherein the step of measuring the expression level of the CI-M6PR gene is a step of measuring the expression level of the translation products.

4. The method as claimed in claim 1, wherein the overexpression of the CI-M6PR gene is determined when the expression of the CI-M6PR gene is at least three times greater than that of said gene in a noncancerous prostate tissue.

5. The method as claimed in claim 3, wherein the step of measuring the expression level of the translation products of the CI-M6PR gene is carried out by analysis of the innnnunohistochennical labeling of the CI-M6PR translation products in a sample of prostate tissue from said subject.

6. The method as claimed in claim 5, wherein the analysis of the innnnunohistochennical labeling of the translation products is evaluated by staining the cells of the sample of prostate tissue, a staining of more than 10% of said cells being an indication of overexpression of the translation products of the CI-M6PR gene.

7. The method as claimed in claim 5, wherein the step of measuring the expression level of the CI-M6PR translation products is carried out using the IgY 415 polyclonal antibody.

8. The method as claimed in claim 1, wherein it also comprises the measurement, in said subject, of the expression level of a basal cell maker or of a cancerous cell marker.

9. An antibody specific for the cation-independent mannose 6-phosphate receptor (CI-M6PR) for use thereof in a method for the in vitro diagnosis of prostate cancer in a subject, said antibody making it possible to measure the expression level of the CI-M6PR gene in a sample of prostate tissue from said subject, the determination of overexpression of said CI-M6PR gene being an indication of the presence of prostate cancer in said subject.

10. The method as claimed in claim 2, wherein the transcription products are the mRNA.

11. The method as claimed in claim 3, wherein the translation products are the CI-M6PR protein.

12. The method as claimed in claim 8, wherein the basal cell marker is chosen from p63, CK 903 or CK 5/6.

13. The method as claimed in claim 8, wherein the cancerous cell marker is chosen from AMACR®, methylated GST1® or TMPRSS2-ETS®.
